(19) United States
(12) Patent Application Publication
(10) Pub. No.: US 2019/0159474 A1
(43) Pub. Date: May 30, 2019

(54) AMELIORATIVE EFFECTS OF A WHOLE COFFEE FRUIT EXTRACT ON AGE-RELATED NEURODEGENERATIVE DISEASE

(71) Applicant: LYTONE ENTERPRISE, INC., New Taipei City (TW)

(72) Inventors: Willian Tienhung CHANG, New Taipei City (TW); Weiting TSENG, New Taipei City (TW); Minghui CHEN, New Taipei City (TW)

(21) Appl. No.: 16/203,772

(22) Filed: Nov. 29, 2018

Related U.S. Application Data
(60) Provisional application No. 62/591,998, filed on Nov. 29, 2017.

(51) Int. Cl. A23F 5/14; A61P 25/28; A23F 5/16; A23F 5/18; A23F 5/24; A61K 36/74

(52) U.S. Cl. CPC A23F 5/14; A61P 25/28; A61K 36/74; A23F 5/18; A23F 5/243; A23F 5/166

(57) ABSTRACT

The invention provides a method of extracting whole coffee fruit, an extract obtained from the method, and method of ameliorating age-related neurodegenerative diseases using said extract. The whole coffee fruit extract may be extracted by water, methanol, ethanol, or acetone and may comprise chlorogenic acid (CA) and procyanidine. The expression levels of p-CREB, BDNF, p-eIF2α, BACE-1, Aβ, NLRP3, caspase-1, IL-1β and COX-2 which may relate to age-related neurodegenerative diseases can be modulated.

### FIELD OF THE INVENTION

The invention provides a method of extracting whole coffee fruit, an extract obtained from the method, and method of ameliorating age-related neurodegenerative diseases using said extract.

### BACKGROUND OF THE INVENTION

Coffee fruits are mainly produced in Africa and the Middle East. Reports indicate that in Africa and Asia, the peel and pulp of coffee fruits are used for fermentation into wines or directly as food for chewing. In Yemen, the peel of coffee fruits is mixed with spices and boiled for making a drink which the local people call “qishr” (Beckman, I. 2000, Journal of the Weston A. Price Foundation). On the other hand, the results of a toxicology test in rats fed with whole coffee fruit extract for 90 days demonstrate that the highest non-observed effect level (NOEL) is 3446 mg/kg bw/day (male rat) and 4087 mg/kg bw/day (female rat) (Heimbach, J. T., et al., Food Chem. Toxicol. 48, 2517-2525). The above references prove that whole coffee fruit is safe for eating and thus can be prepared as food.

Coffee fruits contain high levels of phenol antioxidants. However, in traditional coffee processing, in order to avoid contamination by mycotoxins produced due to rapid decomposition after harvest, the peel and pulp are removed immediately or discarded or used as fertilizer. In fact, the peel and pulp are enriched with nutrients and antioxidants (Napolitana A., et al., J. Agric. Food Chem. 55. 10499-10504). References prove that the water extract of coffee fruits contains more than 85% of polyphenols and that every 100 g extract comprises 0.6 to 1.5 million units of oxygen radical absorption capacity (ORAC) (Nagasawa, H., et al., Anticancer Res. 15. 141-146). Coffee fruits were also proved as having the potential in immune-modulation and inhibiting tumor growth (Kobayashi, T., et al., Anticancer Res. 18. 187-190; Nagasawa, H., et al., Anticancer Res. 15. 141-146).

Aging usually accompanies degeneration in learning memories, which may be caused due to factors such as oxidative stress, neuron cell damage, and neurotrophy factor degeneration. Neurotrophy factors are a family composed of structurally related proteins and can modulate the survival, differentiation, development, and function of neurons of the peripheral and central nervous systems. They are also important for the synapse to modulate neuron signal transduction and function (Huang et al., 2001). Brain-derived neurotrophic factors (BDNF) are the most abundant neurotrophic factors in the brain. Researchers have proven that micro-glial cells secrete large amounts of BDNF and glial cell line-derived neurotrophic factors (GDNF) in damaged brain regions as a mechanism of neuron protection (Imaiet al., 2007; Suzukiet al., 2001). BDNF modulates neurotransmitters, and participates in the processes of neuron growth, differentiation and remodeling, for example, memory and learning controlled by the hippocampus. Protein concentration of BDNF is modulated by neuron activities. When neuron activities increase, the signal transduction pathway of cAMP-response element binding protein (cAMP-CREB) is activated accordingly, wherein CREB is an important transcription factor that enhances the expression level of BDNF (Lonze et al., 2002). The a subunit of eukaryotic initiation factor 2 (eIF2α) is a key subunit during the translation initiation process in eukaryotic organisms, which inhibits the initiation of translation when being phosphorylated, reduces overall protein synthesis, and affects the remodeling of synapses and stabilization of neurons. p-eIF2α also increases activating transcription factor 4 (ATF4) and functions as an inhibitor of CREB, so that subsequent transcription is inhibited. In addition, through gene-specific translation, it can increase the synthesis of BACE-1, drive amyloid precursor protein (APP) to the pathway of forming amyloids, and thus collaboratively causes neuron degeneration.

Over-inflammation will induce deposition of Amyloid-β (Aβ), and Aβ simultaneously activates inflammation reactions, which thus forms a vicious circle. NLRP3 is a member of the NLR family and a complex that detects invasion of foreign micro-organisms and activates inflammation reactions. It converts the cytokine pro-interleukin 1 beta (pro-IL-1β) into an active IL-1β through activating caspase-1 for defending against foreign substances. On the other hand, too much IL-1β in the neuron cells will induce the production of COX-2 and aggravate overall inflammation reactions. Eventually, neuronal inflammation may be caused, leading the neuron to apoptosis.

Alzheimer's disease (AD) is the most common age-related neuron degenerative disease which is characterized by the deposition of Aβ plaques in the brain. Aggregation of Aβ produces large amounts of peroxides, leading to oxidative stress and inflammation and promoting degeneration of neurotrophic factors. Eventually, it may cause death of neuron cells through autophagy or the apoptosis pathway, and causes deficiency in learning and memory abilities (Holtzman et al., 2011).

Over-accumulation of Aβ deposition could induce oxidative stress, inflammatory response, and degeneration of BDNF thereby causing age-related neurodegenerative diseases such as AD. Despite the versatile function of whole coffee fruit extract, it remains unknown with respect to the effect on Aβ deposition and AD.

In the present invention, the effects of a whole coffee fruit extract on neurodegenerative disease and potential protective efficacy against Alzheimer's disease were confirmed. These results indicate that whole coffee fruit extract may have potential therapeutic applications on various neurodegenerative disorders and offer a reference for developing anti-aging health foods in the future.

### SUMMARY OF THE INVENTION

It is found in the present invention that a whole coffee fruit extract is capable of decreasing the expression and deposition of Aβ in the brain, and modulating factors relating to AD. Therefore, the present disclosure provides a novel method for producing a whole coffee fruit extract, a whole coffee fruit extract produced by said method, and a method of ameliorating age-related neurodegenerative diseases, such as Alzheimer's disease, Parkinson's disease, ischemic dementia, and Huntington's disease, using such whole coffee fruit extract.

In a preferred embodiment, the whole coffee fruit extract is extracted by a solvent selected from water, methanol, ethanol, and acetone. In an embodiment, the solvent is ethanol, preferably about 10 to about 50% ethanol, more preferably about 20 to about 40% ethanol, and still more preferably about 25 to about 30% ethanol.

In a preferred embodiment, the whole coffee fruit extract is extracted under the temperature of about 50 to about 100° C. In an embodiment, the temperature is preferably about 60 to about 90° C., and more preferably about 70 to about 80° C.

In a preferred embodiment, the whole coffee fruit extract is extracted for about 0.5 to about 5 hours. In an embodiment, the extraction is preferably conducted for about 3 to about 5 hours, and more preferably for about 3.5 to about 4 hours.

In a preferred embodiment, the whole coffee fruit extract is extracted under a solid to liquid ratio of about 1:20 to about 1:5. In an embodiment, the ratio is selected from about 1:20, about 1:10, and about 1:5, with about 1:10 to about 1:5 being the preferred ratio.

The present disclosure also relates to a whole coffee fruit extract produced by the method described above. In a preferred embodiment, the whole coffee fruit extract comprises 5-O-caffeoylquinic acid (5-CQA), chlorogenic acid (CA) and procyanidine. In an embodiment, the whole coffee fruit extract comprises CA and procyanidine.

The present invention further relates to a method of ameliorating age-related neurodegenerative diseases, comprising administering to a subject in need thereof a therapeutically effective amount of a whole coffee fruit extract described above. In an embodiment, the therapeutic effective amount of the whole coffee fruit extract is about 50 to about 400 mg/kg, preferably about 100 to about 300 mg/kg, more preferably about 200 mg/kg. In an embodiment, Aβ, p-eIF2α, BACE-1, p-CREB, BDNF, NLRP3, caspase-1, IL-1β, and COX-2 in the subject are regulated. In an embodiment, p-eIF2α, BACE-1, Aβ, NLRP3, caspase-1, IL-1β and COX-2 are down-regulated and p-CREB and BDNF are up-regulated. In an embodiment, Aβ deposition in Alzheimer's disease is reduced. In an embodiment, the age-related neurodegenerative diseases are selected from Alzheimer's disease, Parkinson's disease, ischemic dementia, and Huntington's disease. In an embodiment, the age-related neurodegenerative disease is Alzheimer's disease.

The present invention is described in detail in the following sections. Other characterizations, purposes and advantages of the present invention can be easily found in the detailed descriptions and claims of the invention.

### BRIEF DESCRIPTION OF THE DRAWINGS

FIGS. 1A and 1B demonstrate the ratio of expression level of p-eIF2α to β-actin in the hippocampus and cortex, respectively, of 3-month old male SAMP8 mice after feeding with different chews for 12 weeks (a: Control, b: 200 mg/kg whole coffee fruit extract).

FIGS. 2A and 2B demonstrate the ratio of expression level of BACE-1 to β-actin in the hippocampus and cortex, respectively, of 3-month old male SAMP8 mice after feeding with different chews for 12 weeks (a: Control, b: 200 mg/kg whole coffee fruit extract).

FIGS. 3A and 3B demonstrate the percentage of area deposition of Aβ in the hippocampus and whole brain, respectively, of 3-month old male SAMP8 mice after feeding with different chews for 12 weeks (a: Control, b: 200 mg/kg whole coffee fruit extract).

FIGS. 4A and 4B demonstrate the ratio of expression level of p-CREB to CREB in the hippocampus and cortex, respectively, of 3-month old male SAMP8 mice after feeding with different chews for 12 weeks (a: Control, b: 200 mg/kg whole coffee fruit extract).

FIGS. 5A and 5B demonstrate the ratio of expression level of BDNF to β-actin in the hippocampus and cortex, respectively, of 3-month old male SAMP8 mice after feeding with different chews for 12 weeks (a: Control, b: 200 mg/kg whole coffee fruit extract).

FIGS. 6A and 6B demonstrate the ratio of expression level of NLRP3 to β-actin in the hippocampus and cortex, respectively, of 3-month old male SAMP8 mice after feeding with different chews for 12 weeks (a: Control, b: 200 mg/kg whole coffee fruit extract).

FIGS. 7A and 7B demonstrate the ratio of expression level of Caspase-1 p20 to β-actin in the hippocampus and cortex, respectively, of 3-month old male SAMP8 mice after feeding with different chews for 12 weeks (a: Control, b: 200 mg/kg whole coffee fruit extract).

FIGS. 8A and 8B demonstrate the ratio of expression level of IL-1β to β-actin in the hippocampus and cortex, respectively, of 3-month old male SAMP8 mice after feeding with different chews for 12 weeks (a: Control, b: 200 mg/kg whole coffee fruit extract).

FIGS. 9A and 9B demonstrate the ratio of expression level of COX-2 to β-actin in the hippocampus and cortex, respectively, of 3-month old male SAMP8 mice after feeding with different chews for 12 weeks (a: Control, b: 200 mg/kg whole coffee fruit extract).

FIG. 10 shows the results of immunohistochemical (IHC) staining for the levels of Aβ deposition in the brain of 3-month old male SAMP8 mice after feeding with normal chew (A) and the whole coffee fruit extract (200 mg/kg) of the present invention (B) for 12 weeks.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedence over any dictionary or extrinsic definition.

As utilized in accordance with the present disclosure, the following terms, unless otherwise indicated, shall be understood to have the following meanings.

The term “whole coffee fruit” as used herein refers to the entire fruit of the coffee tree (Coffea spec.) in which the exocarp and the outer mesocarp (i.e., the pulp) surround the inner mesocarp (i.e. the mucilage) and endocarp (i.e., the hull), which in turn surround the seeds (i.e., the beans). Thus, the term whole coffee fruit specifically refers to a whole coffee fruit, which may or may not include the seed of the fruit.

The term “whole coffee fruit extract” as used herein refers to the product of any whole coffee fruit extraction process known in the art. Any whole coffee fruit extract is deemed suitable for use herein. For example, a whole coffee fruit extract may be prepared with an aqueous and/or alcoholic solvent to obtain a solution enriched in one or more desirable components (and/or to obtain a material depleted of one or more undesired components). So prepared extracts can further be refined and/or enriched in a specific component using chromatographic methods (e.g., ion exchange, size exclusion, or filtration), or addition of a component or flavoring agent of the whole coffee fruit. Depending on the desired component(s), it should further be recognized that the extracts may also be prepared from selected portions of a coffee cherry (e.g., at least one or more of the bean of the coffee cherry, the pulp, the mucilage, and/or the hull of the whole coffee fruit). In one preferred embodiment, a whole coffee fruit extract is prepared by the method disclosed herein. In another preferred embodiment, the whole coffee fruit extract which is prepared by the method disclosed herein is that under the product name, Anthochlorogin.

The term “ameliorating” as used herein refers to delaying the onset of the symptoms of a susceptible subject or reducing the occurrence of a disease, reducing and/or improving the symptoms of a susceptible subject or increasing the survival rate of the subject with certain lethal disorders or conditions.

The term “age-related neurodegenerative diseases” as used herein refers to the neurodegenerative diseases that progress as age increases, for example, brain function and cognition decline with aging. For example, such diseases include Alzheimer's disease, Parkinson's disease, ischemic dementia, and Huntington's disease.

The term “subject” as used herein denotes animals, especially mammals. In one preferred embodiment, the term “subject” denotes humans.

The term “therapeutically effective amount” as used herein refers to the amount of an active ingredient used alone or in combination with other treatments/medicaments for treating age-related neurodegenerative diseases that shows therapeutic efficacy.

Unless otherwise required by context, singular terms shall include the plural and plural terms shall include the singular.

The inventors of the invention found that a whole coffee fruit extract can enhance the expression of p-CREB and BDNF and decrease the expression of p-eIF2α, BACE-1, Aβ, NLRP3, caspase-1, IL-1β and COX-2. As these parameters may be associated with age-related neurodegenerative disease, the present invention may thus provide a novel therapy for age-related neurodegenerative disease. In a preferred embodiment, the age-related neurodegenerative disease is Alzheimer's disease.

In addition to the data on the enzymes, in situ data showing the reduced deposition of Aβ in the brain of the mice further evidence the efficacy of the whole coffee fruit extract in diseases associated with Aβ deposition. In an embodiment, the disease associated with Aβ deposition is Alzheimer's disease.

Having now generally described the invention, the same may be more readily understood through reference to the following examples, which provide exemplary protocols for the production of the whole coffee fruit extract of the invention and its use in the amelioration of age-related neurodegenerative disease. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLES

### Example 1

### Preparation of Whole Coffee Fruit Extract

Coffee (Coffee Arabica) berry raw materials were obtained from the coffee garden of farmers or agricultural production and marketing groups in Tainan, Pingtung, Hualien and Taitung. The coffee fruits (fresh fruit) were sterilized after receipt so as to reduce surface microbes. Then, the coffee fruits were delivered to the lab by a preservation technology. Right after arrival, the coffee fruits were subjected to cold wind or freeze drying so that the fresh fruits were dried to reach a water content of 20% or less. After that, the coffee fruits were stored or grounded into powder for extraction.

During extraction, the whole coffee fruit powders were dissolved in ethanol for extraction. The extract was subjected to high performance liquid chromatography (HPLC) for analysis of possible active ingredients. The results reveal that the extract comprises 5-CQA, CA and procyanidine. The extract was freeze-dried and a brown to deep brown powder was obtained, which is named as Anthochlorogin. Before evaluation on the biological activities of the extract, the dried product was dissolved in corn oil and adjusted to desired concentrations in the chew.

### Example 2

### Experimental Design and Composition of Feedstuff

Senescence accelerated mice P8 (SAMP8) was used as the animal model for evaluating amelioration of Alzheimer's disease. This mice model is characterized in having age-related memory defects. Whether whole coffee fruit extract has efficacy in improving learning and memory capacities and the effect on related molecular mechanism were explored. 3-month old SAMP8 were raised in 30 (W)×20 (D)×10 (H) cm transparent plastic cages. The temperature was kept at 22±2° C., relative humidity kept at 65±5% and the room had automatically controlled light periods, where 7:00-19:00 was the dark period and 19:00-7:00 was the light period. Before testing, animals were accommodated for 3-5 days. At the beginning of the experiment, mice were separated into a control group taking standard diet (20% Casein, 5% corn oil, 1% vitamin mixture (AIN93-G), 5% mineral mixture (AIN93-G), 2% cellulose powder, and 2.5% choline) and an experimental group taking an additional 200 mg/kg whole coffee fruit extract. Said 200 mg/kg is a human daily dose and was converted to mice dose for administration. Feedstuff and water were taken freely. During the experiment, the amounts of food intake and body weight were recorded. After feeding for 12 weeks, the mice were sacrificed and brain samples were subjected to analysis for amyloid β (Aβ), phospho-eukaryotic initiation factor 2α (p-eIF2α), beta-site amyloid precursor protein-cleaving enzyme-1 (BACE-1), brain-derived neurotrophic factor (BDNF) and phospho-cAMP response element-binding protein (p-CREB). Levels of the inflammation markers NLRP3, caspase-1, IL-1β, and COX2 were also measured.

### Example 3

### Western Blot Analysis

Brain tissue samples were homogenized. According to the molecular weight of the target protein to be observed, different concentrations of SDS-PAGE gels were prepared for electrophoresis. The samples were heated at 100° C. and sequentially loaded into the wells of the SDS-PAGE gels. Separation was conducted at 65V 100 mins and 100V 80 mins for the target protein to be able to run to the desired position. After electrophoresis, SDS-PAGE gels and PVDF membranes were placed in a cassette for transferring the target protein. The transfer was conducted in a 4° C. cold room.

After completion of transfer, the PVDF membrane was cut according to the molecular weight of the target protein and placed in small box for blocking for 1 hour. The membrane was then washed by wash buffer on a shaker for several times. After that, primary antibody solution was added to soak the membrane on a shaker in a 4° C. cold room and recovered after 8-16 hours of incubation. Secondary antibody solution was then added to soak the membrane on a shaker at room temperature for 1 hour. The PVDF membrane was then incubated with an enhanced chemiluminescence (ECL) substrate and placed in a chemiluminescence imaging system for taking photos of the images.

The data of each experimental group in this example were analyzed by SPSS software. The results are represented by mean±S.E.M. Differences of protein expression among each group were analyzed using one-way analysis of variance (one-way ANOVA). P<0.05 represents significant difference.

### Example 3.1

### Analysis of the Expression Level of the Aβ Related Factor p-eIF2α

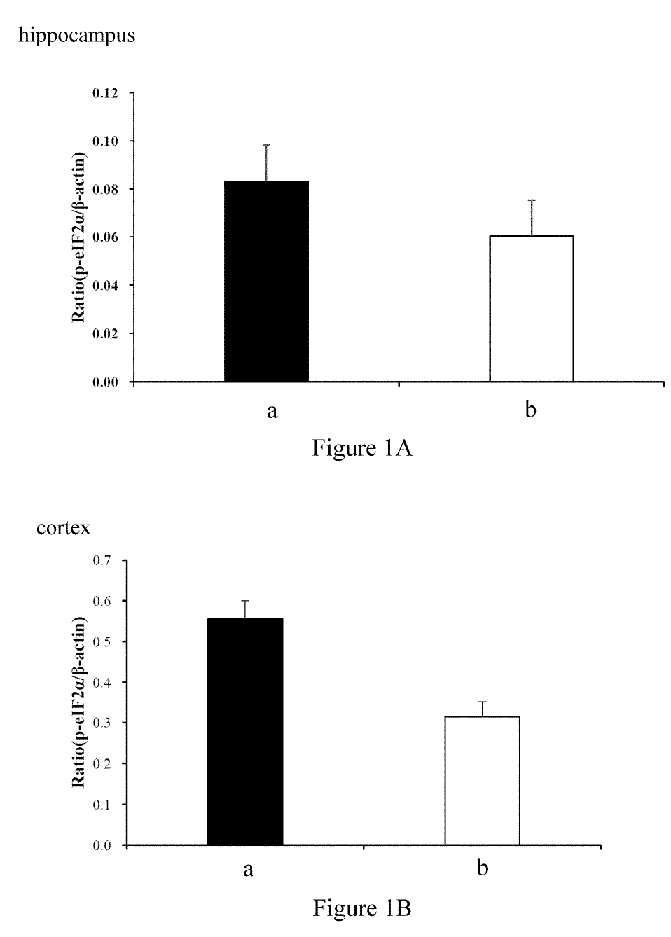

As shown in FIGS. 1A and 1B, in 3-month old male SAMP8 mice fed with different chews (a: Control, b: 200 mg/kg whole coffee fruit extract) for 12 weeks, the ratio of expression levels of p-eIF2α to β-actin in the hippocampus (FIG. 1A) and cortex (FIG. 1B) of the mice brain are decreased. The decreases reach statistical significance.

### Example 3.2

### Analysis of the Expression Level of Aβ Regulatory Factor BACE-1

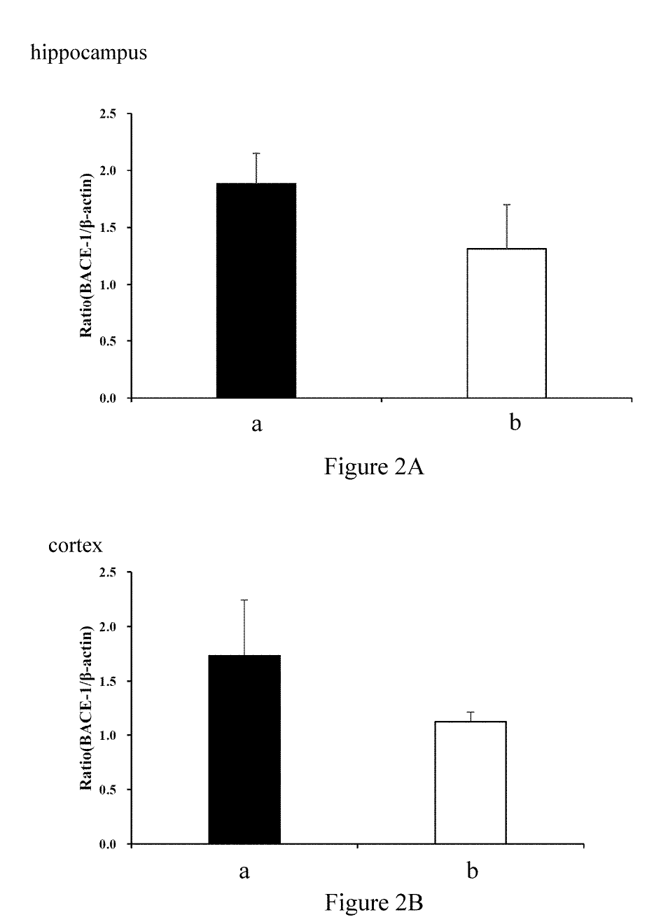

As shown in FIGS. 2A and 2B, in 3-month old male SAMP8 mice fed with different chews (a: Control, b: 200 mg/kg whole coffee fruit extract) for 12 weeks, the ratio of expression levels of BACE-1 to β-actin in the hippocampus (FIG. 2A) and cortex (FIG. 2B) of the mice brain are decreased. The decreases reach statistical significance.

### Example 3.3

### Analysis of the Deposition of Aβ in the Brain

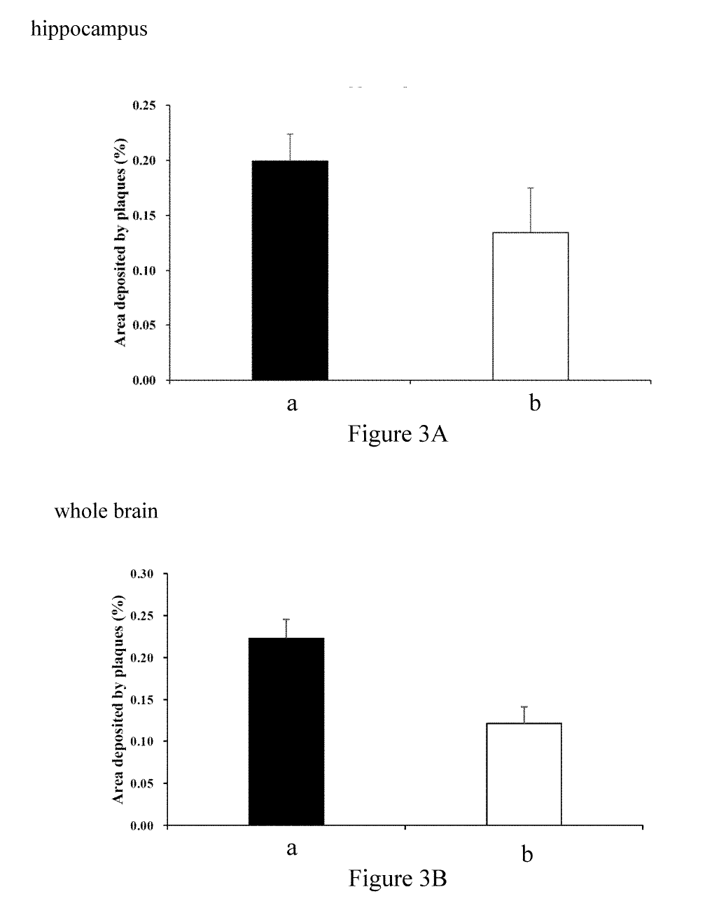

As shown in FIGS. 3A and 3B, in 3-month old male SAMP8 mice fed with different chews (a: Control, b: 200 mg/kg whole coffee fruit extract) for 12 weeks, the percentage of area deposition of Aβ in the hippocampus (FIG. 3A) and whole brain (FIG. 3B) of the mice brain are decreased. The decreases reach statistical significance.

### Example 3.4

### Analysis of the Expression Level of Neurotrophy Factor p-CREB

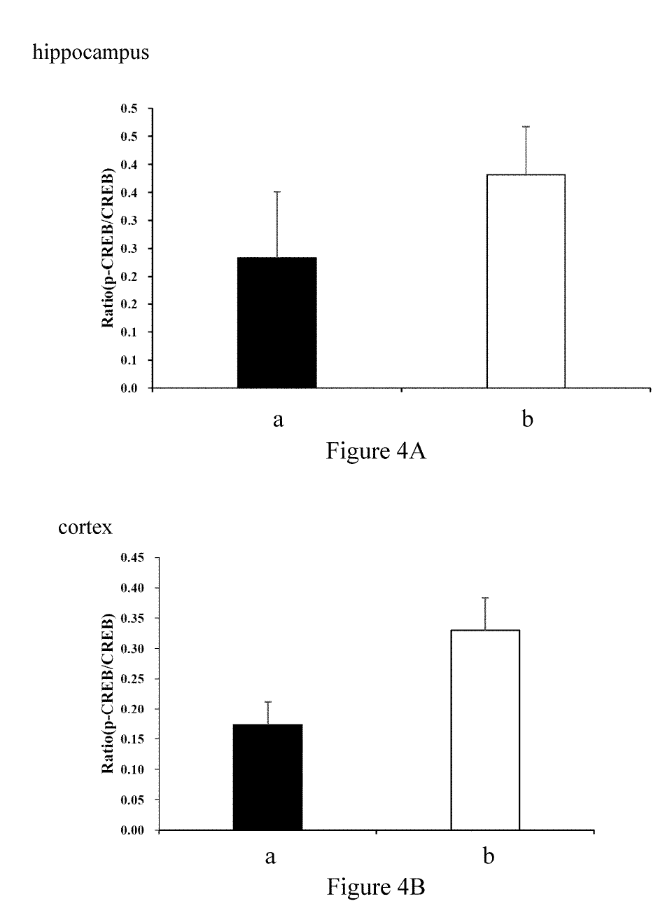

As shown in FIGS. 4A and 4B, in 3-month old male SAMP8 mice fed with different chews (a: Control, b: 200 m/kg whole coffee fruit extract) for 12 weeks, the ratio of expression levels of p-CREB to CREB in the hippocampus (FIG. 4A) and cortex (FIG. 4B) of the mice brain are increased. The increases reach statistical significance.

### Example 3.5

### Analysis of the Expression Level of Neurotrophy Factor BDNF

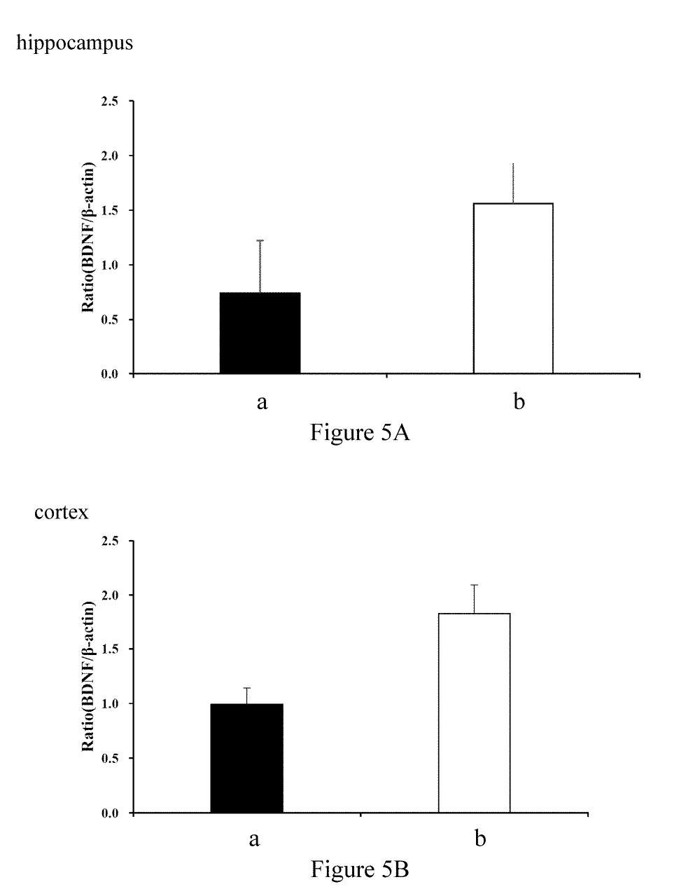

As shown in FIGS. 5A and 5B, in 3-month old male SAMP8 mice fed with different chews (a: Control, b: 200 m/kg whole coffee fruit extract) for 12 weeks, the ratio of expression levels of BDNF to β-actin in the hippocampus (FIG. 5A) and cortex (FIG. 5B) of the mice brain are increased. The increases reach statistical significance.

### Example 3.6

### Analysis of the Expression Level of Inflammation Related Factor NLRP3

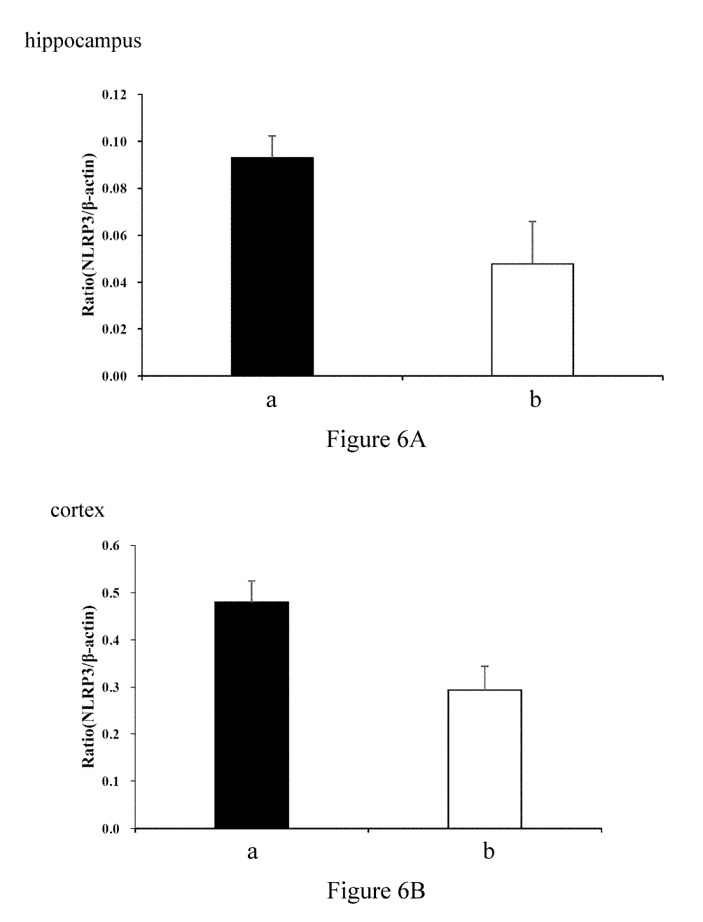

As shown in FIGS. 6A and 6B, in 3-month old male SAMP8 mice fed with different chews (a: Control, b: 200 m/kg whole coffee fruit extract) for 12 weeks, the ratio of expression levels of NLRP3 to β-actin in the hippocampus (FIG. 6A) and cortex (FIG. 6B) of the mice brain are decreased. The decreases reach statistical significance.

### Example 3.7

### Analysis of the Expression Level of Inflammation Related Factor Caspase-1

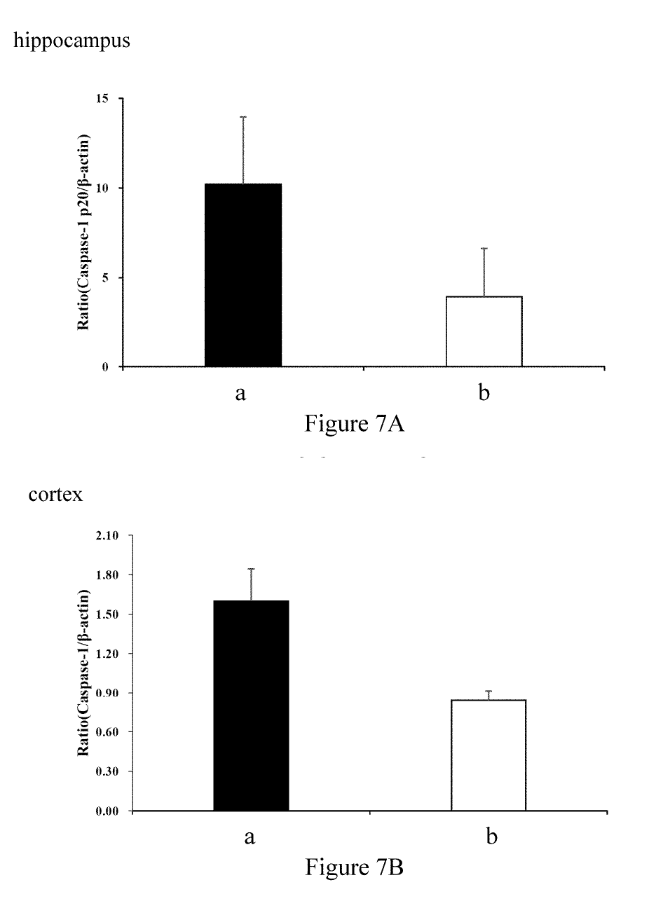

As shown in FIGS. 7A and 7B, in 3-month old male SAMP8 mice fed with different chews (a: Control, b: 200 m/kg whole coffee fruit extract) for 12 weeks, the ratio of expression levels of caspase-1 p 20 to β-actin in the hippocampus (FIG. 7A) and cortex (FIG. 7B) of the mice brain are decreased. The decreases reach statistical significance.

### Example 3.8

### Analysis of the Expression Level of Inflammation Related Factor IL-1β

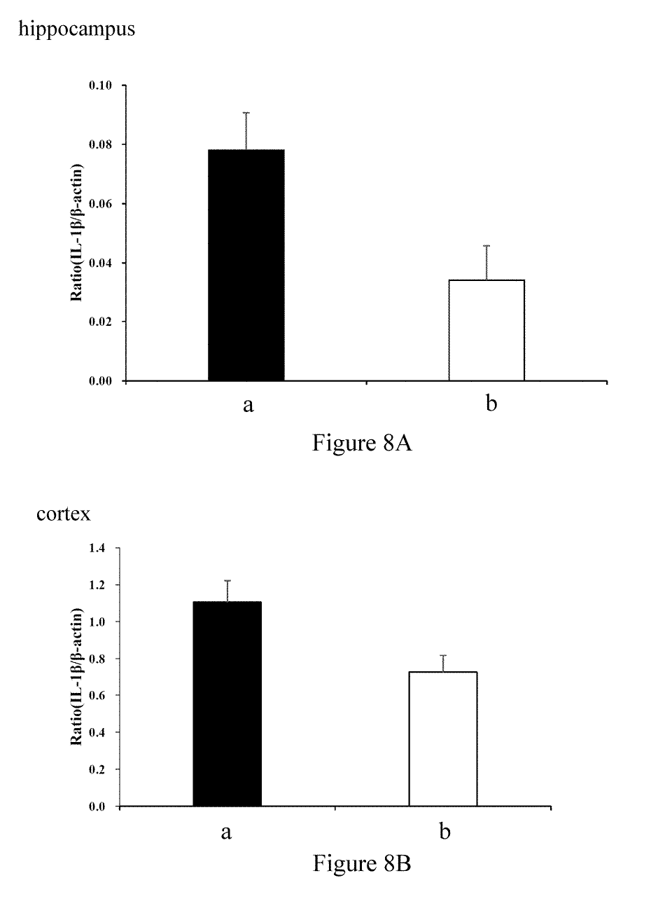

As shown in FIGS. 8A and 8B, in 3-month old male SAMP8 mice fed with different chews (a: Control, b: 200 m/kg whole coffee fruit extract) for 12 weeks, the ratio of expression levels of IL-1β to β-actin in the hippocampus (FIG. 8A) and cortex (FIG. 8B) of the mice brain are decreased. The decreases reach statistical significance.

### Example 3.9

### Analysis of the Expression Level of Inflammation Related Factor COX-2

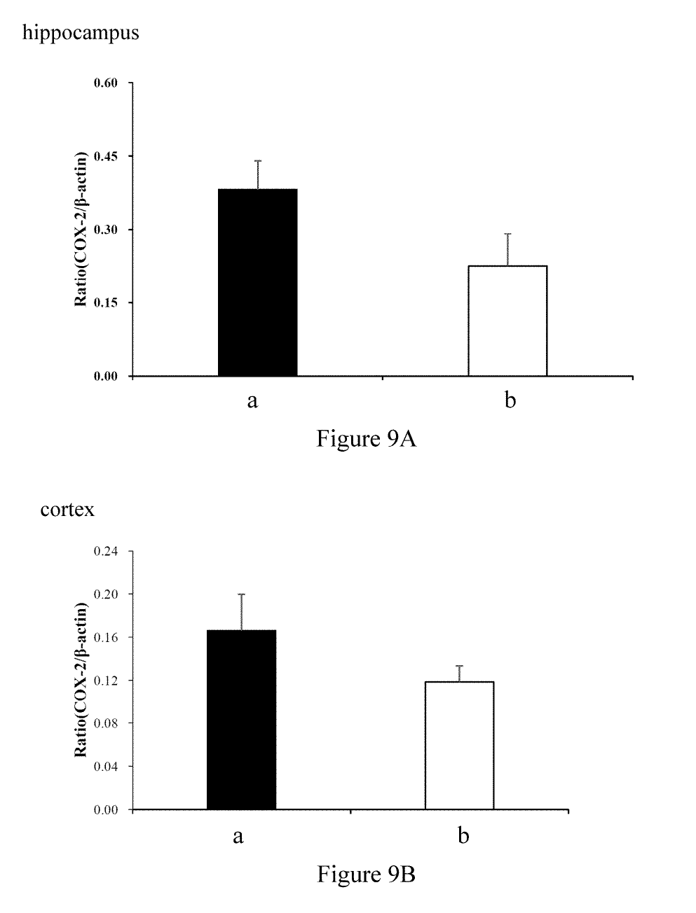

As shown in FIGS. 9A and 9B, in 3-month old male SAMP8 mice fed with different chews (a: Control, b: 200 m/kg whole coffee fruit extract) for 12 weeks, the ratio of expression levels of COX-2 to β-actin in the hippocampus (FIG. 9A) and cortex (FIG. 9B) of the mice brain are decreased. The decreases reach statistical significance.

### Example 4

### Immunohistochemical (IHC) Staining for Aβ

After mice were sacrificed, brain tissues were obtained and immediately soaked in 10% formalin for paraffin embedding and tissue slicing (3-5 μm/slice). Before performing IHC staining, the slices were placed in xylene for dewaxing and treated with ethanol for removing xylene. The slices were soaked in Trilogy and heated at 121° C. for 15 minutes to restore antigenicity followed by IHC staining. At first, goat serum-PBS solution was added for blocking. After that, diluted monoclonal anti-Aβ antibody was added and incubated at 4° C. overnight. The next day, superenhancer solution was added for reaction at room temperature in a dark place followed by reaction with poly-HRP reagent at room temperature in the dark place. DAB was used for 1.5 minutes for colorification and then hematoxylin was used for counter stain. After completion, the slices were placed on an optical microscope to observe the density of a brownish substance which is equivalent to the amount of Aβ deposition.

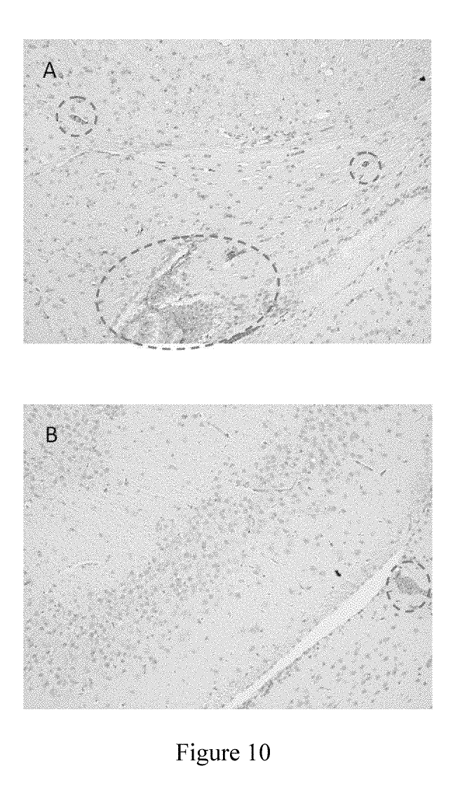

The results are shown in FIG. 10 which represents the level of deposition of Aβ in the brain of 3-month old male SAMP8 mice fed with different diets for 12 weeks. FIG. 10A is the result of the control group in which mice were fed with normal chew. FIG. 10B is the result of the experimental group in which mice were fed with chew additionally added with 200 mg/kg whole coffee fruit extract of the present invention. The results in FIG. 10 clearly demonstrate that the level of Aβ deposition in FIG. 10A is obviously more intense than that in FIG. 10B. This result indicates that mice fed with 200 m/kg whole coffee fruit extract of the present invention have significantly reduced amount of Aβ deposition in the brain of SAMP8 mice. Thus, diseases or symptoms associated with Aβ deposition may be ameliorated in SAMP8 mice fed with the whole coffee fruit extract of the present invention.

Age-related neurodegenerative diseases may be caused due to oxidative stress, neuron cell damage, or decreased levels of neurotrophy factors. From the examples demonstrated above, it was surprisingly found that by ingesting the whole coffee fruit extract of the present invention, the levels of neurotrophy factors p-CREB and BDNF were increased, and p-eIF2α and BACE-1 that regulate Aβ were decreased. Eventually, decreased deposition of Aβ was observed. Further, the expression levels of inflammation factors NLRP3, caspase-1, IL-1β and COX-2 were decreased. Thus, the whole coffee fruit extract of the present invention, which may comprise chlorogenic acid (CA) and procyanidine, may be useful for ameliorating age-related neurodegenerative diseases.

Numerous modifications and variations of the invention as set forth in the above illustrative examples are expected to occur to those skilled in the art. Consequently, only such limitations as appear in the appended claims should be placed on the invention.

What is claimed is:

1. A method of producing a whole coffee fruit extract, comprising the steps of:
(a) providing freshly collected whole coffee fruit, optionally drying the whole coffee fruit so that the water content is 20% or less;
(b) subjecting the dried whole coffee fruit of step (a) to extraction by a solvent selected from water, methanol, ethanol, and acetone; and
(c) recovering the extract of step (b).

2. The method of claim 1, wherein the extraction in step (b) is conducted under the temperature of about 50 to about 100° C.

3. The method of claim 1, wherein the extraction in step (b) is conducted for about 0.5 to about 5 hours.

4. The method of claim 1, wherein the solid to liquid ratio in the extraction in step (b) is about 1:20 to about 1:5.

5. The method of claim 1, wherein the extraction in step (b) is performed by about 25 to about 30% ethanol at a solid to liquid ratio of about 1:10 to about 1:5 at about 70 to about 80° C. for about 3.5 to about 4 hours.

6. A whole coffee fruit extract obtained from the method of claim 1.

7. The whole coffee fruit extract of claim 6, comprising chlorogenic acid (CA) and procyanidine.

8. A method of ameliorating age-related neurodegenerative diseases, comprising administering to a subject in need thereof a therapeutically effective amount of a whole coffee fruit extract.

9. The method of claim 8, wherein the expression levels of p-CREB and BDNF are increased and the expression levels of p-eIF2α, BACE-1, Aβ, NLRP3, caspase-1, IL-1β and COX-2 are decreased.

10. The method of claim 9, wherein Aβ deposition in Alzheimer's disease is reduced.

11. The method of claim 8, wherein the age-related neurodegenerative diseases are selected from Alzheimer's disease, Parkinson's disease, ischemic dementia, and Huntington's disease.

12. The method of claim 11, wherein the age-related neurodegenerative disease is Alzheimer's disease.

13. The method of claim 8, wherein the therapeutically effective amount of the whole coffee fruit extract is about 50 to about 400 mg/kg.

14. The method of claim 13, wherein the therapeutically effective amount of the whole coffee fruit extract is about [symbol] 200 m/kg.
